(19) United States
(12) Patent Application Publication
(10) Pub. No.: US 2018/0368994 A1
(43) Pub. Date: Dec. 27, 2018

(54) Expanding Spinal Implant

(71) Applicant: Quandary Medical, LLC, Denver, CO (US)

(72) Inventors: Jeffrey R. Schell, Denver, CO (US); Brandon Arthurs, Wilmington, NC (US); Ryan Arce, Denver, CO (US); Scott Noble, Denver, CO (US); Leighton Lapierre, Denver, CO (US)

(73) Assignee: Quandary Medical, LLC, Denver, CO (US)

(21) Appl. No.: 15/991,783

(22) Filed: May 29, 2018

Related U.S. Application Data
(63) Continuation of application No. 15/791,241, filed on Oct. 23, 2017.
Application No. 15/791,241 is a continuation of application No. 15/862,257, filed on Jan. 4, 2018.
(60) Provisional application No. 62/511,913, filed on May 26, 2017.
(60) Provisional application No. 62/569,746, filed on Oct. 9, 2017.
(60) Provisional application No. 62/639,677, filed on Mar. 7, 2018.
(60) Provisional application No. 62/442,356, filed on Jan. 4, 2017.

(51) Int. Cl. A61F 2/46; A61F 2/44; A61B 17/56; A61B 17/00

(52) U.S. Cl. CPC A61F 2/4611; A61F 2/4455; A61B 17/56; A61F 2002/4677; A61B 2017/00022; A61F 2/4644; A61B 2017/564; A61F 2/4684

(57) ABSTRACT

The preferred embodiment of the present invention is described as an expandable spinal implant. Generally, the inventor intends for the expandable spinal implant to function as an implant that translates compressive force into anterior-posterior and vertical force to enable the implant to both distract and expand between two endplates of adjacent vertebral bodies, optionally into a lordotic profile.

This application claims benefit to provisional patent application No. 62/511,913, entitled “Multidimensional Poplif”, filed May 26, 2017, which is incorporated by reference in its entirety for all purposes. This application claims benefit to provisional patent application No. 62/569,746, entitled “Neuromonitored Dilation System”, filed Oct. 9, 2017 which is incorporated by reference in its entirety for all purposes. This application claims benefit to nonprovisional patent application Ser. No. 15/791,241, entitled “System and Method for Spinal Surgery Utilizing a Low-Diameter Sheathed Portal Shielding an Oblique Lateral Approach Through Kambin's Triangle,” filed Oct. 23, 2017, which is incorporated by reference in its entirety for all purposes. This application claims benefit to provisional patent application No. 62/639,677, entitled “Expanding Surgical Portal”, filed Mar. 7, 2018, which is incorporated by reference in its entirety for all purposes.

### BACKGROUND OF THE INVENTION

The present inventors have recognized the yet unsolved problem of a spine implant fitting through a low diameter portal (in some cases as low as 8 mm diameter or less), and then expanding both vertically and horizontally into a lordotic profile. Other proposed solutions for the problem of a spine implant fitting through a 8 mm portal and then expanding both vertically and horizontally into a lordotic profile may exist in the prior art, but such solutions have proven inadequate. For instance, expansion in both the vertical and horizontal dimensions remains an unsolved problem, as does the footprint surface area to enable sufficient contact with a vertebral endplate. Other solutions have failed to provide an adequate void for the placement of bone graft, facilitating fusion of the inferior and superior vertebral bodies via bone healing through the implant. In prior art expandable implant solutions, the expansion of the implant places too much stress upon the arms of the implant, thereby causing structural failure. In prior art solutions, the positioning of the expandable device into the ideal location within the disc space is difficult. In prior art solutions, making sure the expandable implant is fully extended beyond the opening of the access portal is difficult. In prior art solutions, the implant assembly is associated with instability, as it accompanies a high degree of “toggle” or “slop” between the components. In prior art solutions, the expandable implant may become trapped resulting from partial deployment within the disc space. Prior art solutions are also difficult to remove following placement.

### BRIEF DESCRIPTION OF FIGURES

FIG. 1 depicts an embodiment of the expandable spinal implant in its compressed form.

FIG. 2 depicts an alternative embodiment of the expandable spinal implant in its compressed form.

FIG. 3 depicts a view of the proximal end of the preferred embodiment of the expandable spinal implant in its compressed form.

FIG. 4 depicts a view of the distal end of the preferred embodiment of the expandable spinal implant in its compressed form.

FIG. 5 depicts an alternative embodiment of the expandable spinal implant in its compressed form.

FIG. 6 depicts a view of the distal end of the preferred embodiment of the expandable spinal implant in its deployed form.

FIG. 7 depicts a top down view of the preferred embodiment of the expandable spinal implant in its deployed form.

FIG. 8 depicts a bottom up view of an embodiment of the expandable spinal implant in its deployed form.

FIG. 9 depicts a compressive end and links of an embodiment of the expandable spinal implant in its deployed form.

FIG. 10 depicts a perspective view of the interaction between a compressive end, a link and a connecting beam of an embodiment of the invention in its deployed form.

FIG. 11 depicts a perspective view of the distal end of an embodiment of the invention highlighting the atraumatic conical tip.

FIG. 12 depicts a perpendicular view of the interaction between a link and a connecting beam connected by a pivot pin in an embodiment of the invention.

FIG. 13 depicts the connective ends and center stem in an embodiment of the invention.

FIG. 14 depicts a perspective view of a link as utilized in an embodiment of the invention.

FIG. 15 depicts a perpendicular view of a connecting beam featuring gripping teeth in an embodiment of the invention.

FIG. 16 depicts an embodiment of the center stem.

FIG. 17 depicts a cross-sectional view of an embodiment of the invention.

FIG. 18 depicts an embodiment of the center stem.

### SUMMARY OF THE INVENTION

The preferred embodiment of the present invention is described as an expandable spinal implant. Generally, the inventor intends for the expandable spinal implant to function as an low profile implant that transits through a low diameter sheath or portal in its compressed form, with mechanisms to subsequently translate compressive force into anterior-posterior and vertical force to enable the implant to both distract and expand between two endplates of adjacent vertebral bodies, optionally into a lordotic profile, in its deployed form. In such configuration, the expandable spinal implant may be placed between two vertebral bodies and subsequently expanded from a compressed form into a deployed form.

### DETAILED DESCRIPTION OF THE INVENTION

In its preferred embodiment, the expandable spinal implant is configured such that it may transition from a compressed form having a lower diameter (in the preferred embodiment, 9 millimeters or less) into a deployed form having a larger expanded footprint in its deployed form. Once the expandable spinal implant is in its deployed form, the expandable spinal implant may then subsequently be locked to ensure stability by mechanisms readily apparent by those skilled in the art, such as by the placement of a locking screw.

In varying embodiments, the implant is placed through a portal, such as that described in provisional patent application No. 62/639,677, entitled “Expanding Surgical Portal”, filed Mar. 7, 2018, which is incorporated by reference in its entirety for all purposes. The present inventors intend for the expandable spinal implant to be placed in any variety of spinal fusion procedures, but specifically intend for it to be utilized in association with the oblique lateral lumbar interbody fusion (OLLIF) procedure, and more precisely the version of the OLLIF procedure described within nonprovisional patent application Ser. No. 15/791,241, entitled “System and Method for Spinal Surgery Utilizing a Low-Diameter Sheathed Portal Shielding an Oblique Lateral Approach Through Kambin's Triangle,” filed Oct. 23, 2017, which is incorporated by reference in its entirety for all purposes. The present inventors contemplate that the expandable spinal implant may be placed during a procedure using any variety of known spinal instrumentation, including that more precisely described in nonprovisional patent application Ser. No. 15/862,257 entitled “System for Approaching the Spine Laterally and Retracting Tissue in an Anterior to Posterior Direction,” which is incorporated by reference in its entirety for all purposes.

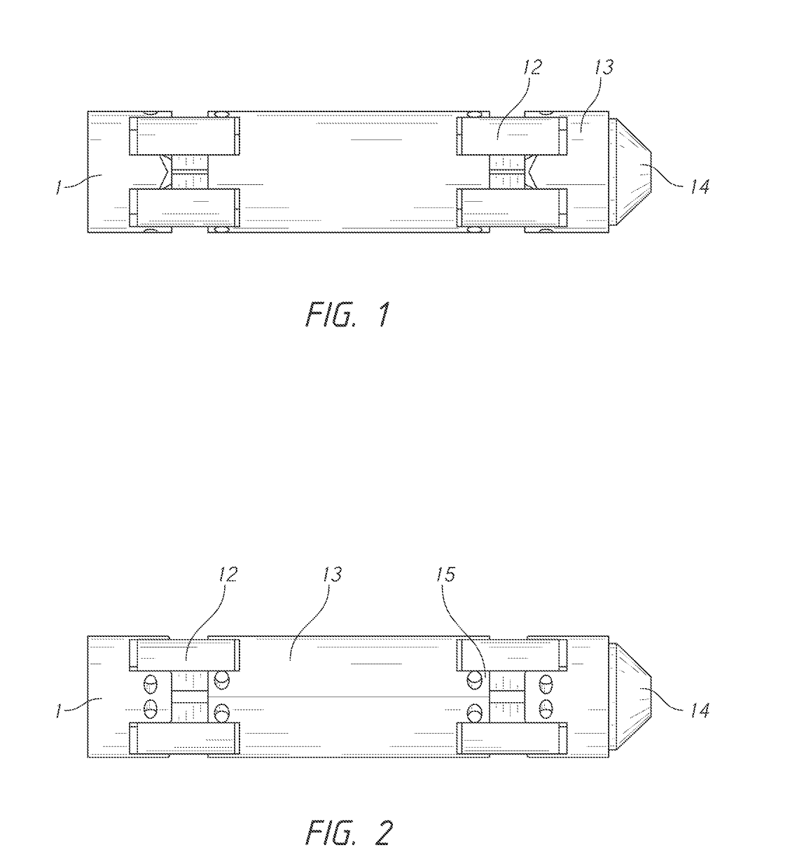

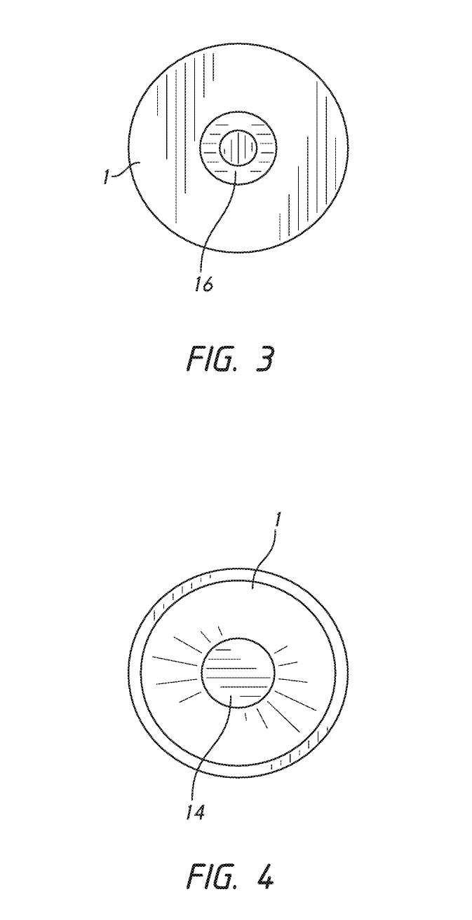

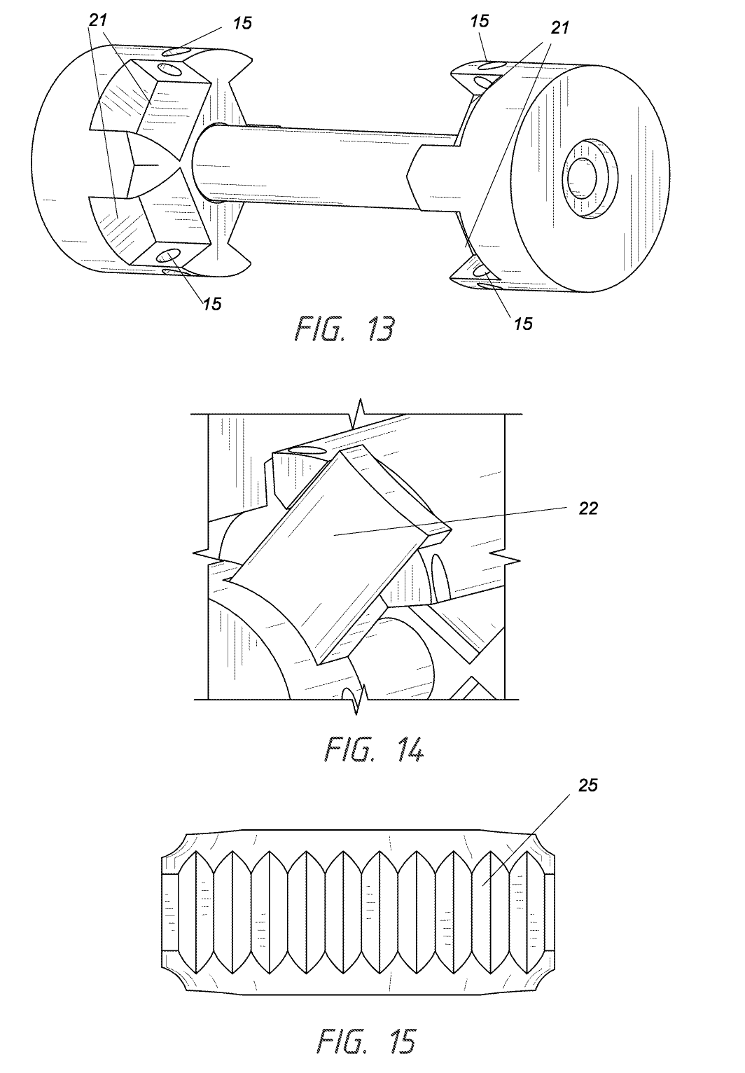

An embodiment of the invention incorporates one or more compressive ends 1. A single compressive end is depicted within FIG. 1 in an embodiment of the invention. In the preferred embodiment of the invention, the one or more compressive ends 1 are 9 mm in diameter. The preferred embodiment of the compressive ends 1 incorporates medical grade titanium as the primary material in its composition. A compressive end 1 in an embodiment of the invention is described as cylindrically-shaped component with a center hole to accept a center stem 16, depicted in FIG. 18. In an embodiment, the compressive ends 1 also comprise four cut outs 21, as depicted in FIG. 13, to mate with a link 22, as depicted in FIG. 14. In varying embodiments, a link may comprise a 45 degree link, configured to be oriented at a 45 degree angle off of the horizontal plane, or 30 degree link 12, configured to be oriented at a 30 degree angle off of the horizontal plane. In the preferred embodiment, the configuration of the one or more links 22 relative to the one or more compressive ends 1 and the one or more connecting beams 13 allows over top dead center configuration while the embodiment of the invention is in the fully deployed position. The compressive ends 1 in an embodiment of the invention are also described as having one or more pivot pin holes 15 each configured to accept a pivot pin 24, fixing the one or more links 22 to the one or more compressive ends also comprising pivot pin holes 15, and the one or more connecting beams 13 also comprising pivot pin holes 15, 1 via mechanisms and processes known to those skilled in the art. In alternative embodiments of the invention incorporating two links 22 and two connecting beams 13, the compressive ends 1 only incorporate two pivot pin holes 15 designed to enable interaction with the two links 22.

An embodiment of the invention incorporates a plurality of pivot pin holes 15, a sub-component of links 22 associated with embodiments of the invention. In the preferred embodiment of the invention, each link 22 consists of a 30 degree link 12, as depicted by FIG. 6. In the preferred embodiment, each pivot pin hole 15 has a diameter of 1 millimeter. In the preferred embodiment, the pivot pin hole 15, comprises a void within a surrounding Medical Grade Titanium piece in its composition. A pivot pin hole 15 in an embodiment of the invention is described as a through hole allowing for the placement of a pivot pin 24 through the related components, and thereby enabling rotational motion of the surrounding part that the pivot pin hole 15 exists within about said pivot pin 24.

In an embodiment of the invention, a pivot pin hole 15 and a compressive end 1 are related, as the pivot pin hole 15 cut into a portion of the compressive end 1.

An embodiment of the invention incorporates a 30 degree link 12, as depicted by FIG. 6. The preferred embodiment of the 30 degree link 12 comprises the following dimensions: 7 mm tall by 3.5 mm wide by 2.5 mm deep. The preferred embodiment of the 30 degree link 12, incorporates medical grade titanium in its composition. A 30 degree link 12 in an embodiment of the invention is described as a component fixedly attached to both the compressive end 1 and the connecting beam 13 by welded pins, such that the when seen from a back view, the 30 Degree Links are 30 degrees from the horizontal plane, as depicted in FIG. 6. A 30 degree link 12 in an embodiment of the invention is also described as having the ability to movably rotate about one or more pivot pins 24, such that the one or more compressive ends 1 are able to freely move away one another or toward each other along the center stem 16. A 30 degree link 12 in an embodiment of the invention is also described as comprising a geometry that conforms to the mating geometries of both the one or more compressive ends 1 and the one or more connecting beams 13, such that when the expandable spinal implant is fully deployed, the one or more 30 degree links 12 exist in what those skilled in the arts know to be over top dead center of the two ends of the expandable spinal implant, optionally comprising compressive ends 1.

In an embodiment of the invention, a 30 degree link 12 and one of the ends of the expandable spinal implant, optionally comprising a compressive end 1 are related. In an embodiment, the 30 degree link 12 and a compressive end 1 are related to one another in such embodiment by the 30 degree link connects to the compressive end 1 by pivot pin attachment, which connects the 30 degree link 12 and one of the compressive ends 1 by placement of the pivot pin 24 through each related component's pivot pin hole 15.

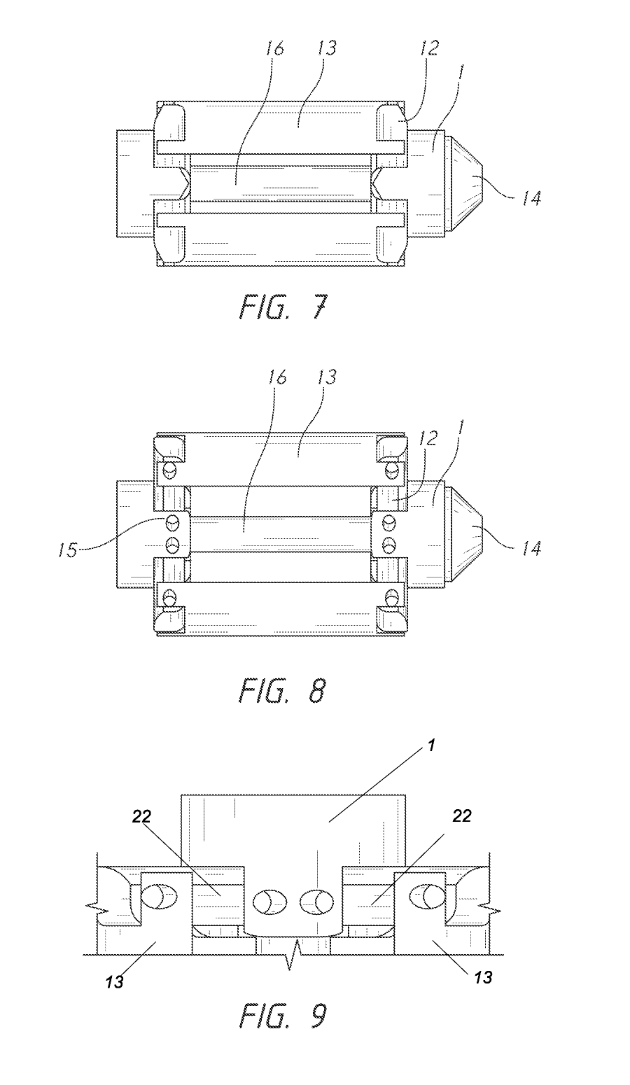

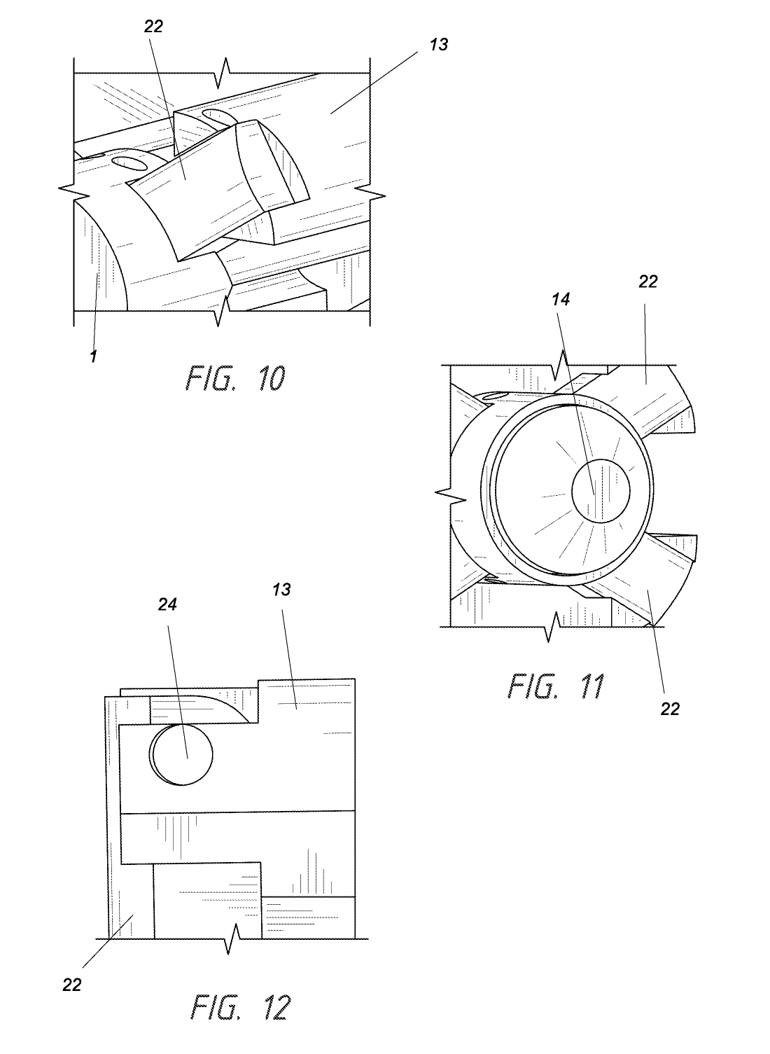

An embodiment of the invention incorporates at least two connecting beams 13, as depicted by FIG. 7. In the preferred embodiment of the invention there are four connecting beams, as depicted in FIG. 6. In the preferred embodiment, a connecting beam 13 exists to the following dimensions: a quadrant of a 9 millimeter diameter cylinder, with a length of 20 millimeters. In an alternative embodiment, the connecting beam 13 comprises the following dimensions, a half of a 9 millimeter diameter cylinder. In varying embodiments, gripping teeth 25 exist on the exterior surface (the surface most distal to the center stem) of the connecting beam, as depicted in FIG. 15. The preferred embodiment of the connecting beam 13, incorporates medical grade titanium in its composition. A connecting beam 13 in an embodiment of the invention is described as an elongated cylindrical quadrant which spans the distance from the plurality of links 22. A connecting beam 13 in an embodiment of the invention is also described as containing bone grabbing gripping teeth 25 along the curved side. A connecting beam 13 in an embodiment of the invention is also described as having cutouts conforming to the shape of and intended to accept links 22, including the 45 or 30 Degree Links, as demonstrated in FIG. 8. A connecting beam 13 in an embodiment of the invention is also described as having one or more pivot pin holes 15 to accept one or more pivot pins 24. In the preferred embodiment, the one or more pivot pins 24 travel through the pivot pin holes 15 of both at least one connecting beam 1 and at least one link 22, thereby connecting the two components in a fashion that allows each component to rotate around the one or more pivot pins 24.

An embodiment of the invention incorporates a pivot pin hole 15, a sub-component of a 30 degree link 12, as depicted by FIG. 8. The preferred embodiment of the pivot pin hole 15 comprises the following dimensions: 1 mm in diameter. The preferred embodiment of the pivot pin hole 15, comprises a void through the surrounding component of the expandable spinal implant. A pivot pin hole 15 in an embodiment of the invention is described as a through hole allowing assembly of one or more Pivot Pins 19 through the related components, and rotational motion about said one or more Pivot Pins 19.

An embodiment of the invention incorporates a center stem 16, as depicted by FIG. 7. The preferred embodiment of the center stem 16 comprises the following dimensions: 29 mm long and 3 mm in diameter. Another embodiment of the center stem 16 comprises the dimensions of 8 mm in diameter at the largest section of the atraumatic conical tip 14. A center stem 16 in an embodiment of the invention is described as hollow elongated cylinder with the distal end comprising an atraumatic conical tip 14, and the proximal end comprising female threading located within the hollow section.

A center stem 16 and at least one compressive end 1 are related to one another in an embodiment as the center stem 16 is fixedly welded to at least one compressive end 1 at or near the atraumatic conical tip 14.

In the preferred embodiment, the atraumatic conical tip 14 comprises the following dimensions: 8 mm in diameter at its widest point. Another embodiment of the atraumatic conical tip 14 comprises a 45 degree taper from the narrowest point at the distal end, as depicted in FIG. 7 and FIG. 11. Another embodiment of the atraumatic conical tip 14 comprises the dimensions of a 3 mm extrusion. The preferred embodiment of the atraumatic conical tip 14, incorporates Medical Grade Titanium in its composition. An atraumatic conical tip 14 in an embodiment of the invention is described as a tapered conical extrusion at the distal most end of the center stem 16.

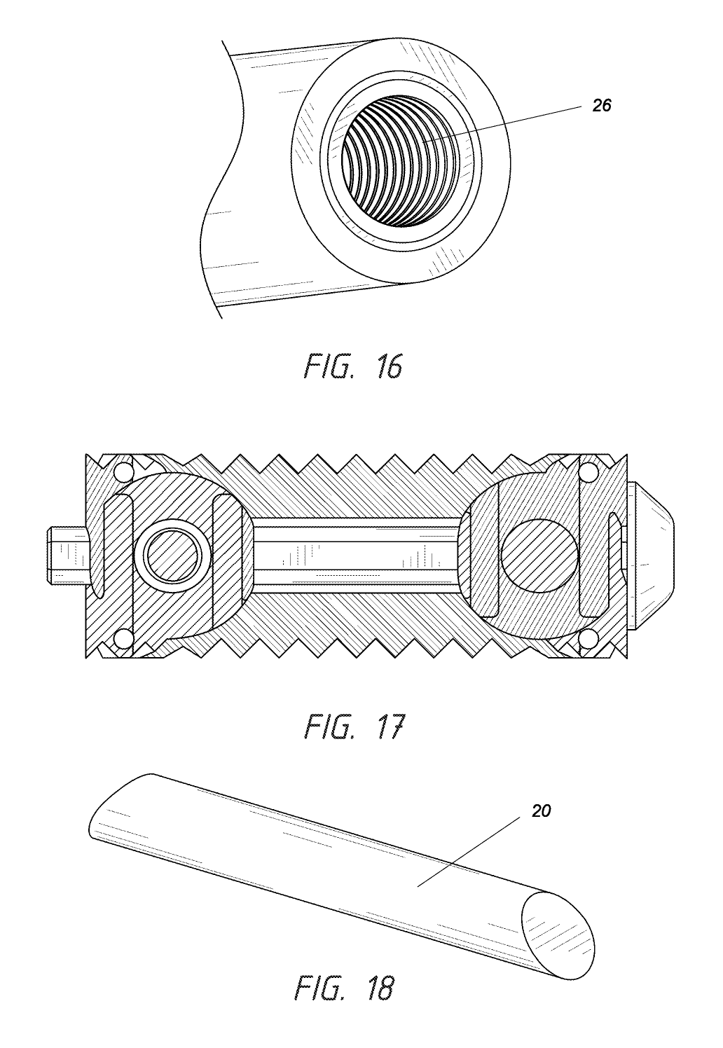

An embodiment of the invention incorporates threads 26, preferably female threads, as a sub-component of a center stem 16, as depicted by FIG. 16. The threads 26 in an embodiment of the invention are more precisely described as female threading located within the hollow proximal end of the center stem 16. The center stem 16 having threads 26 is configured such that it can link via the threads 26 to a wire able to be pulled by a surgeon during surgery to effectuate the pulling of the distal compressive end, optionally with a mechanism to place a pushing force on the proximal surface of the proximal compressive end, to thereby compress the two ends together and transition the preferred embodiment of the expandable spinal implant from its compressed for into its deployed form. In such embodiment, the threads 26 can also subsequently accommodate a locking screw to provide stability to the expandable spinal implant in its deployed form by providing a static compressive force via mechanisms apparent to those skilled in the art.

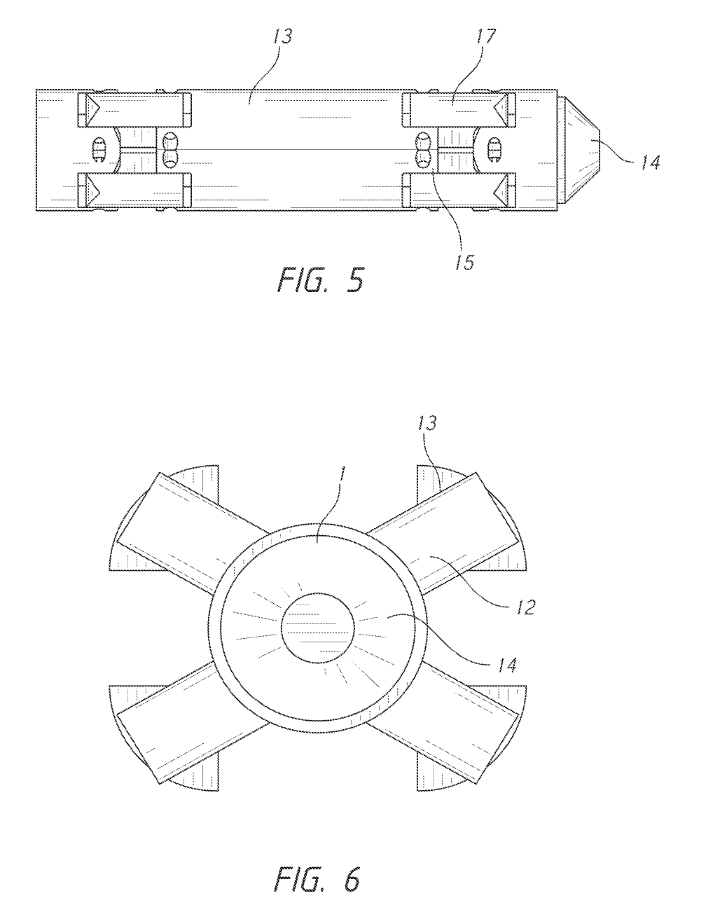

An embodiment of the invention incorporates a 45 degree link, as depicted by FIG. 5. The preferred embodiment of the 45 degree link 17 comprises the following dimensions: 7 mm tall by 3.5 mm wide by 2.5 mm deep. The preferred embodiment of the 45 degree link 17, incorporates Medical Grade Titanium in its composition. A 45 degree link 17 in an embodiment of the invention is described as a component fixedly attached to both at least one compressive end 1 and at least one connecting beam 13 by one or more pivot pins 24. A 45 degree link 17 in an embodiment of the invention is also described as having rotational motion about the one or more pivot pins 24 such that the compressive ends 1 are able to freely elongate and shorten the distance in relation to each other along the center stem 16. A 45 degree link 17 in an embodiment of the invention is also described as comprising a geometry that conforms to the mating geometries of both the plurality of compressive ends 1 and the plurality of connecting beams 13, such that when the expandable spinal implant is fully deployed, the 45 Degree Links exist in what those skilled in the arts know to be over top dead center with respect to the one or more compressive ends 1.

In an embodiment of the invention, a 45 degree link 17 and a connecting beam 13 are related. A 45 degree link 17 and a connecting beam 13 are related to one another in such embodiment by the 45 degree link relates to the connecting beam by a pivot pin attachment, whereby a pivot pin 19 slides through the pivot pin holes 15 located in each related component.

In an embodiment of the invention, a 45 degree link 17 and compressive ends 1 are related. A 45 degree link 17 and a compressive ends 1 are related to one another in such embodiment by the 45 degree link relates to the compressive ends by pivot pin attachment, whereby a pivot pin 19 slides through the pivot pin holes 15 located in each related component.

In an embodiment of the invention, a 45 degree link 17 and a pivot pin hole 15 are related. Moreover, the pivot pin hole 15 is a sub-component of a 45 degree link 17 in such embodiment.

An embodiment of the invention incorporates a pivot pin hole 15, a sub-component of a 30 degree link 12, as depicted by FIG. 15. The preferred embodiment of the pivot pin hole 15 comprises the following dimensions: 1 mm in diameter. A pivot pin hole 15 in an embodiment of the invention is described as a through hole allowing assembly of one or more Pivot Pins 19 through the related components, and rotational motion about said one or more Pivot Pins 19.

An embodiment of the invention incorporates a endplate mesh. In such embodiment, the endplate mesh exists between two connecting beams to spread the load of the endplate from the point of interaction between the connecting beams and the endplate. The endplate mesh expands or stretches to bridge the void between two connecting beams in the plane that is parallel to and along a vertebral body endplate. In the preferred embodiment of the invention incorporating endplate mesh, the endplate mesh is comprised of titanium or nitonol.

The preferred embodiment of the invention incorporates a void for bone graft. Those skilled in the art recognize the desirability of having a void to place biologic material, such as bone graft, within the implant, so that once bone heals together during the process of spinal fusion, the bone heals through the spinal implant, thereby strengthening the overall stability of the fused construct. A void for bone graft in an embodiment of the invention is described as space for the bone graft to fill into. In an embodiment of the invention, bone graft is deposited into the void by the compressive ends acting in a plunger mechanism as the compressive force pushes the ends of the implant inward. In an alternative embodiment, bone graft is placed after the expandable spinal implant is placed in situ in deployed form by subsequently placing bone graft during surgery.

An embodiment of the invention incorporates a shallow-angle implant 6. A shallow-angle implant 6 in an embodiment of the invention is described as an implant featuring end links arrayed at less than a 90 degree angle to the vertically adjacent end link. This allows for lower distraction height implants.

An embodiment of the invention incorporates one or more pivot pins 24, as depicted by FIG. 12. The preferred embodiment of the pivot pin 24 is configured to have a diameter of 1 millimeter. The preferred embodiment of the pivot pin 24, incorporates titanium in its composition. Alternatively the pivot pin 24 incorporates medical grade stainless steel in its composition. A pivot pin 24 in an embodiment of the invention is described as a small dowel that mates into corresponding Pivot Pin Holes 15 within an embodiment of the invention.

In the foregoing specification, specific embodiments have been described. However, one of ordinary skill in the art appreciates that various modifications and changes can be made without departing from the scope of the invention as set forth in the claims below. One of ordinary skill in the art also appreciates specifically that a variety of substitute materials could be utilized in each of the inventive components without departing from the scope of the invention as set forth in the claims below. Accordingly, the specification and figures are to be regarded in an illustrative rather than a restrictive sense,

and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The terms “coupled” and “linked” as used herein is defined as connected, although not necessarily directly and not necessarily mechanically. A device or structure that is “configured” in a certain way is configured in at least that way, but may also be configured in ways that are not listed. Also, the sequence of steps in a flow diagram or elements in the claims, even when preceded by a letter does not imply or require that sequence.

I claim:

1. An expandable spinal implant for placement between two vertebral bodies and subsequent expansion from a compressed form into a deployed form, comprising:
at least one compressive end;
a plurality of connecting beams;
a plurality of links; and
a plurality of pivot pins.

2. The expandable spinal implant of claim 1, further comprising a center stem.

3. The expandable spinal implant of claim 2, the center stem further comprising threading.

4. The expandable spinal implant of claim 1, the plurality of links configured to expand at a 30 degree angle relative to the horizontal plane.

5. The expandable spinal implant of claim 1, the plurality of links configured to expand at a 45 degree angle relative to the horizontal plane.

6. The expandable spinal implant of claim 1, configured such that in its compressed form its diameter is no greater than 9 millimeters.

7. The expandable spinal implant of claim 1, the plurality of connecting beams further comprising gripping teeth.

8. The expandable spinal implant of claim 1, configured such that at least one of the plurality of links is over top dead center with respect to the at least one compressive end.
